(19) United States
(12) Patent Application Publication
(10) Pub. No.: US 2016/0151000 A1
(43) Pub. Date: Jun. 2, 2016

(54) TRUNK MUSCLE CONTRACTION DETECTION APPARATUS

(71) Applicant: Murata Manufacturing Co., Ltd., Nagaokakyo-shi (JP)

(72) Inventor: TORU SHIMUTA, Nagaokakyo-shi (JP)

(21) Appl. No.: 15/016,329

(22) Filed: Feb. 5, 2016

Related U.S. Application Data
(63) Continuation of application No. PCT/JP2014/068866, filed on Jul. 16, 2014.

(30) Foreign Application Priority Data
Aug. 7, 2013 (JP) 2013-163951

(51) Int. Cl. A61B 5/11; A61B 5/08; A61B 5/00; A61B 5/024

(52) U.S. Cl. CPC A61B 5/1107; A61B 5/02405; A61B 5/0816; A61B 5/7207; A61B 5/7278

(57) ABSTRACT

The occurrence of trunk muscle contraction associated with body movement and the like is accurately and timely detected using pulse data.

A trunk muscle contraction detection apparatus includes a change-component acquisition unit that extracts change-component data representing a change component of pulse-interval data, the change component regarding pulse intervals, a vibration-component removing unit that generates vibration-component removal data by removing, from the change-component data, a vibration component corresponding to periodic vibrations in the pulse-interval data, and a variation-component extraction unit that extracts a certain variation component from the vibration-component removal data, and determines the occurrence of trunk muscle contraction in accordance with the certain variation component, which has been extracted.

### CROSS REFERENCE TO RELATED APPLICATIONS

The present application is a continuation of PCT/JP2014/068866 filed Jul. 16, 2014, which claims priority to Japanese Patent Application No. 2013-163951, filed Aug. 7, 2013, the entire contents of each of which are incorporated herein by reference.

### FIELD OF INVENTION

The present invention relates to a trunk muscle contraction detection apparatus.

### BACKGROUND

Hitherto, a technology for detecting a movement or a state of a human body by sensing feature values regarding the human body has been known. For example, Patent Document 1 discloses a configuration for determining a movement of a human body by analyzing an output from an acceleration sensor mounted in a portable device. Feature values acquired from the acceleration sensor of a portable device attached to a human body are compared with reference data, and body movements such as swinging arms, walking, and the like are recognized.

In addition, Patent Document 2 discloses a configuration for subjecting heartbeat intervals to frequency analysis and estimating a sleeping state, an autonomic nervous state (a state of fatigue), or a breathing state from the size of a frequency band component characteristic of sleep depth, autonomic nervous (sympathetic nervous, parasympathetic nervous) activity, or breathing.

Patent Document 1: Japanese Unexamined Patent Application Publication No. 2002-65640.

Patent Document 2: Japanese Unexamined Patent Application Publication No. 2003-290164.

However, with the configuration of Patent Document 1, movements of a portion to which the acceleration sensor is attached are detected. Thus, when the portable device is in the state of being held by a hand, movements of portions other than the hand are unable to be detected. In addition, body movements that do not involve a displacement such as deep breathing and straining are not detected because an acceleration is not applied to the acceleration sensor by such body movements. In contrast, in the case where an acceleration is applied to the acceleration sensor because of a factor other than body movements such as traveling in a vehicle, a determination error may occur when it is presumed that a body movement is present.

In addition, with the configuration of Patent Document 2, since analysis is performed in accordance with detected pulses, inconveniences that arise when an acceleration sensor is used are solved such as there being limited portions that can be detected, non-detection of body movements involving no displacement, and the effect of an acceleration caused by a movement other than body movements. However, with the configuration of Patent Document 2, in the case where heartbeat periodic variation caused by a factor that is a body movement and heartbeat periodic variation caused by a factor that is autonomic nervous activity are present in the same frequency range, variations caused by these factors are not distinguished from each other. In addition, about a few minutes of data is needed to perform frequency analysis, and thus it takes a long time to acquire an analysis result. In the first place, autonomic nerve analysis requires, as a precondition, acquisition of static data in a resting state, and a heart-rate variation component caused by an instant body movement is to be a target that is removed as noise, and thus autonomic nerve analysis is not suitable for timely body movement detection.

### SUMMARY OF THE INVENTION

Thus, an object of the present invention is to provide a trunk muscle contraction detection apparatus and method that is capable of accurately and timely detecting the occurrence of trunk muscle contraction associated with a body movement and the like using pulse data.

A trunk muscle contraction detection apparatus according to the present invention includes a processor or CPU constituting a change-component acquisition unit that acquires change-component data representing a change component of pulse-interval data, the change component regarding pulse intervals, a vibration-component removing unit that generates vibration-component removal data by removing, from the change-component data, a vibration component corresponding to periodic vibrations in the pulse-interval data, and a variation-component extraction unit that extracts a certain variation component from the vibration-component removal data, and determines the occurrence of trunk muscle contraction in accordance with the certain variation component, which has been extracted.

According to the trunk muscle contraction detection apparatus according to the present disclosure, a variation component caused by the trunk muscle contraction is extracted from data obtained by removing, from the change-component data of the pulse-interval data, a vibration component caused by, for example, respiratory variation, and thus it becomes possible to assuredly detect trunk muscle contraction. Although trunk muscle contraction is also caused by breathing, periodic breathing is treated as noise, and thereafter variations caused by trunk muscle contraction are distinguished from respiratory variation.

In the trunk muscle contraction detection apparatus according to the present invention, preferably, the certain variation component is a component corresponding to a change waveform in which an upward peak has appeared immediately after a downward peak, in a waveform representing the pulse-interval data.

In this case, a change in pulse interval that is characteristic of the time of trunk muscle contraction is extracted as the variation component from the pulse-interval data, and thus it becomes possible to determine trunk muscle contraction with high accuracy.

In the trunk muscle contraction detection apparatus according to the present invention, preferably, the vibration component is a component based on respiratory variation.

In this case, even in the case where the respiratory variation is large in the pulse-interval data, a change component caused by the respiratory variation is assuredly removed as the vibration component and it becomes possible to determine trunk muscle contraction with high accuracy.

In the trunk muscle contraction detection apparatus according to the present invention, the change-component acquisition unit includes an interpolation processing unit that interpolates the pulse-interval data, arranges pieces of data of the pulse-interval data at constant time intervals, and supplies the interpolated pulse-interval data to the vibration-component removing unit.

In this case, interpolation of the pulse-interval data makes it possible to cut out the pulse-interval data at constant time intervals, and thus it becomes possible to process the pulse-interval data in a frequency domain. Thus, for example, an adaptive filter may be used in the vibration-component removing unit, and thus the degree of freedom of a data processing configuration is improved. In addition, it becomes easier to capture vibration characteristics by performing an interpolation process on the pulse-interval data having a short vibration period, and thus it becomes possible to improve the accuracy of vibration component removal.

The trunk muscle contraction detection apparatus according to the present invention includes pulse-interval data generation unit that detects pulses of a subject and generates the pulse-interval data.

According to the trunk muscle contraction detection apparatus according to the present invention, pulses are detected, and it becomes possible to accurately and timely detect trunk muscle contraction in accordance with the detection output.

According to the present invention, it is possible to accurately and timely detect the occurrence of trunk muscle contraction associated with body movement and the like using pulse data.

### BRIEF DESCRIPTION OF DRAWINGS

FIG. 1 is a block diagram illustrating a trunk muscle contraction detection apparatus according to a first embodiment.

FIG. 2 is a diagram for describing the relationship between a pulse interval and a trunk muscle movement.

FIG. 3 is a diagram for describing respiratory vibration.

FIG. 4 is a diagram for describing a vibration-component removal process in a trunk muscle contraction detection method according to the first embodiment.

FIG. 5 is a diagram for describing the vibration-component removal process of FIG. 4 in a supplemental manner.

FIG. 6 is a flowchart illustrating the trunk muscle contraction detection method according to the first embodiment.

FIG. 7 is a block diagram illustrating a trunk muscle contraction detection apparatus according to a second embodiment.

FIG. 8 is a flowchart illustrating a trunk muscle contraction detection method according to the second embodiment.

FIG. 9 is a block diagram illustrating a trunk muscle contraction detection apparatus according to a third embodiment.

FIG. 10 is a flowchart illustrating a trunk muscle contraction detection method according to the third embodiment.

### DETAILED DESCRIPTION

In the following, preferred embodiments of the present invention will be described in detail with reference to the drawings. Note that the same elements are denoted by the same reference numerals in the drawings, and redundant description thereof will be omitted.

### First Embodiment

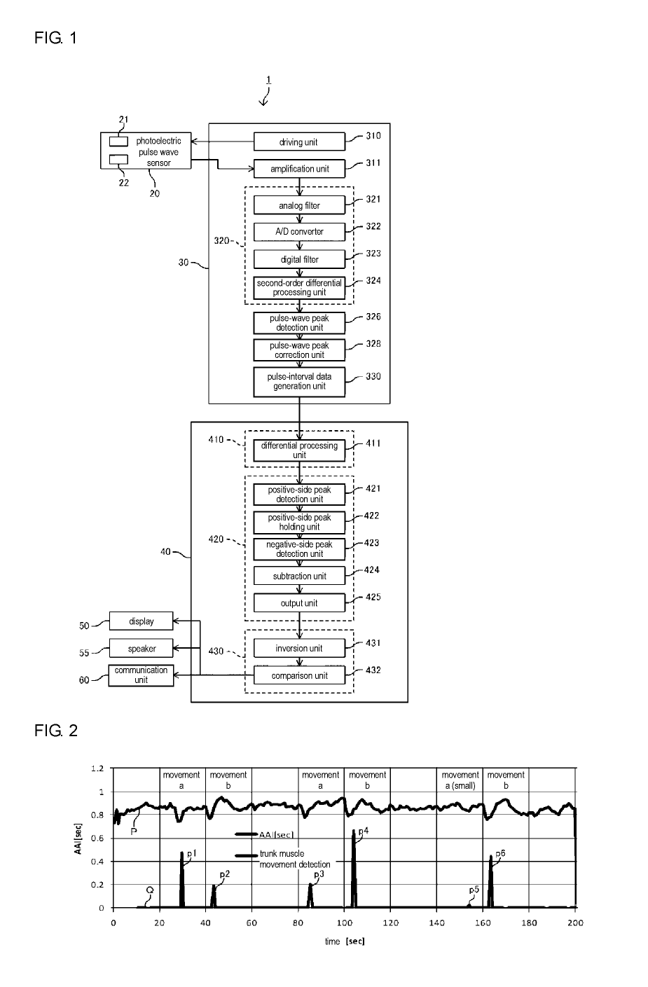

First, the configuration of a trunk muscle contraction detection apparatus 1 according to a first embodiment will be described using FIG. 1. FIG. 1 is a block diagram illustrating the configuration of the trunk muscle contraction detection apparatus 1. The trunk muscle contraction detection apparatus 1 of the present embodiment acquires change-component data regarding pulse intervals in pulse-interval data, generates vibration-component removal data by removing a component corresponding to periodic vibrations from the change-component data, and determines the occurrence of trunk muscle contraction by extracting a certain variation component from the vibration-component removal data. In the following, structural elements will be described in detail.

The trunk muscle contraction detection apparatus 1 includes a pulse sensor 20 for acquiring pulses, a pulse-interval data acquisition unit 30 for generating pulse-interval data, and a pulse-interval data analysis unit 40 for performing analysis to detect trunk muscle contraction in accordance with detected pulse-interval data. In the following, the structural elements will be described in detail. Note that the pulse sensor 20 and the pulse-interval data acquisition unit 30 can be constituted by the processor and/or CPU provided to generate the pulse-interval data as described herein.

The pulse sensor 20 may be, for example, a photoelectric pulse wave sensor for detecting pulses, an electrocardiogram sensor for detecting heartbeats, or a piezoelectric sensor. Note that, herein, pulses and heartbeats are collectively called pulses. The pulse sensor 20 may be a wearable sensor that may be attached to a human body, or may also be a grip-type sensor such as a game controller or a smartphone that may be held by a hand. In the present embodiment, description will be made supposing that, as an example, the pulse sensor 20 includes a photoelectric pulse wave sensor. Note that the pulse sensor 20 (hereinafter also referred to as “photoelectric pulse wave sensor 20”) is a sensor that optically detects a photoelectric pulse wave signal using light absorption characteristics of hemoglobin in blood. Thus, the photoelectric pulse wave sensor 20 includes a light emitting element 21 and a light receiving element 22.

The light emitting device 21 emits light in response to a pulsed driving signal output from a driving unit 310 of the pulse-interval acquisition unit 30. For example, a light-emitting diode (LED), a Vertical Cavity Surface Emitting LASER (VCSEL), or a resonator-type LED, or the like may be used as the light emitting element 21. Note that the driving unit 310 generates and outputs a pulsed driving signal that drives the light emitting element 21.

The light receiving element 22 outputs a detection signal based on the intensity of light that has been emitted from the light emitting element 21, that has passed through, for example, a human body such as a fingertip or has been reflected by a human body, and then that enters the light receiving element 22. For example, a photodiode or a phototransistor may be preferably used as the light receiving element 22. A photodiode is used as the light receiving element 22 in the present embodiment. The light receiving element 22 is connected to the pulse-interval acquisition unit 30, and a detection signal (a photoelectric pulse wave signal) acquired by the light receiving element 22 is output to the pulse-interval acquisition unit 30.

The pulse-interval acquisition unit 30 acquires pulse intervals by processing an input photoelectric pulse wave signal, and generates pulse-interval data. Thus, the pulse-interval acquisition unit 30 includes an amplification unit 311, a signal processing unit 320, a pulse-wave peak detection unit 326, a pulse-wave peak correction unit 328, and a pulse-interval data generation unit 330. In addition, the signal processing unit 320 includes an analog filter 321, an A/D converter 322, a digital filter 323, and a second-order differential processing unit 324.

Here, among the above-described units, the digital filter 323, the second-order differential processing unit 324, the pulse-wave peak detection unit 326, and the pulse-wave peak correction unit 328 are constituted by, for example, a CPU or microprocessor that performs computational processing, a ROM that stores programs and data for causing the CPU to execute processes, and a RAM that temporarily stores various types of data such as a computation result. That is, the functions of the above-described units are realized by the CPU executing the programs stored in the ROM. It should further be appreciate that other units as described herein can be constituted by the CPU or an additional CPU to execute processes to perform the algorithms described herein.

As described above, the signal processing unit 320 includes the analog filter 321, the A/D converter 322, the digital filter 323, and the second-order differential processing unit 324, and extracts a pulse component by performing a filtering process on a photoelectric pulse wave signal amplified by the amplification unit 311.

The analog filter 321 and the digital filter 323 removes components (noise) other than frequencies that characterize a photoelectric pulse wave signal, and performs filtering to improve the S/N. To be more specific, since a frequency component of about 0.1 to a few tens of hertz is predominant in a photoelectric pulse wave signal, the S/N is improved by performing a filtering process using an analog filter and a digital filter such as a low pass filter and a band pass filter and by causing only signals of the above-described frequency range to selectively pass through the analog filter and the digital filter.

Note that both the analog filter 321 and the digital filter 323 do not have to be provided, and only either of the analog filter 321 and the digital filter 323 may be provided. Note that the photoelectric pulse wave signal on which the analog filter 321 and the digital filter 323 have performed the filtering process is output to the second-order differential processing unit 324.

The second-order differential processing unit 324 acquires a second-order differential pulse wave (acceleration pulse wave) signal by subjecting the photoelectric pulse wave signal to second-order differentiation. The acquired acceleration pulse wave signal is output to the pulse-wave peak detection unit 326. Note that, for a photoelectric pulse wave peak, a change (a rising edge) may not be obvious, and it may be difficult to detect the photoelectric pulse wave peak. Thus, it is preferable in the exemplary embodiment that the photoelectric pulse wave peak be converted into an acceleration pulse wave and peak detection be performed. However, the second-order differential processing unit 324 does not have to be provided and may be omitted according to an alternative embodiment.

The pulse-wave peak detection unit 326 detects a peak (rising edge) of a photoelectric pulse wave signal (acceleration pulse wave) on which the filtering process has been performed by the signal processing unit 320. According to the exemplary embodiment, the pulse-wave peak detection unit 326 saves, in a RAM or the like, information on, for example, a peak time and a peak amplitude about all detected peaks.

The pulse-wave peak correction unit 328 calculates a delay time for a photoelectric pulse wave signal at the signal processing unit 320 (the analog filter 321, the digital filter 323, and the second-order differential processing unit 324). The pulse-wave peak correction unit 328 corrects, in accordance with the calculated delay time for the photoelectric pulse wave signal, a peak of the photoelectric pulse wave signal (acceleration pulse wave signal) detected by the pulse-wave peak detection unit 326. The corrected peak of the photoelectric pulse wave (acceleration pulse wave) is output to the pulse-interval data generation unit 330.

The pulse-interval data generation unit 330 generates pulse-interval data by collecting corrected peaks of a photoelectric pulse wave, and outputs the generated pulse-interval data to the pulse-interval data analysis unit 40. An example of pulse-interval data is illustrated in FIG. 2. In FIG. 2, the horizontal axis represents time and the vertical axis represents pulse interval (AAI). Data P represents pulse intervals (AAI), and data Q represents trunk muscle contraction detection results. Here, a pulse interval is a value obtained by dividing a unit time by the number of pulses. For example, in the case where the number of pulses for 60 seconds is 80, a pulse interval is 0.75 (=60/80). As shown by the data P, although the pulse interval randomly changes, the pulse interval steeply drops at timings at which trunk muscle contraction occurs, and then increases. For example, in the case of a subject shown by data of the graph of FIG. 2, at timings at which a movement A (a trunk muscle contraction movement for swinging arms) and a movement B (a trunk muscle contraction movement for crouching forward) are performed, steep drops and recovery (rises) of the pulse interval are observed. The trunk muscle contraction detection results represented by the data Q will be described later.

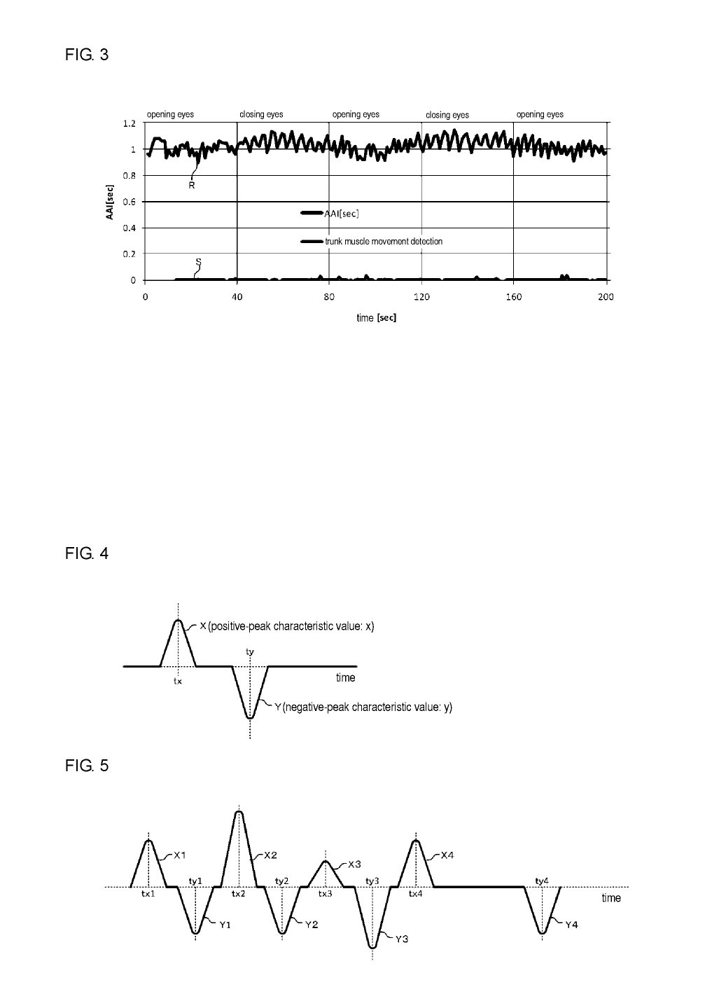

Trunk muscle contraction is detected by extracting such steep drops and rises of the pulse interval in the present embodiment. In the behavior of the pulse interval, regular changes, namely, periodic changes such as respiratory variation are also observed in addition to random changes and steep changes that occur at the times of occurrence of trunk muscle contraction as shown by the data P. For example, FIG. 3 illustrates respiratory variations of pulse intervals.

In FIG. 3, the horizontal axis represents time and the vertical axis represents pulse interval (AAI). Data R represents pulse intervals (AAI), and data S represents trunk muscle contraction detection results. Note that the subject shown by the pulse-interval data of FIG. 2 (hereinafter referred to as “subject A”) and a subject shown by pulse-interval data of FIG. 3 (hereinafter referred to as “subject B”) are different persons. Although changes in pulse interval caused by respiratory variation vary between individuals, it is known that a range of frequency is from about 0.1 to 0.5 Hz. As understood from FIGS. 2 and 3, the subject A's trunk muscle contraction is relatively obviously seen, but the subject B's trunk muscle contraction is buried in respiratory variations and difficult to detect (note that the subject B is only breathing, and trunk muscle contraction does not occur in FIG. 3). Consequently, it is necessary to assuredly distinguish, in acquired changes in pulse interval, steep changes caused by trunk muscle contraction from changes caused by respiratory variation.

The pulse-interval data analysis unit 40 includes a change-component acquisition unit 410, a vibration-component removing unit 420, and a variation-component extraction unit 430. The pulse-interval data analysis unit 40 is configured to extract an increase in pulse interval after pulse interval reduction, the increase occurring in a short period of time in pulse-interval data.

The change-component acquisition unit 410 includes a differential processing unit 411 in the present embodiment. The differential processing unit 411 differentiates, using a certain number of pulses as a differential interval, pulse-interval data in which pulse interval values are arranged in time sequence. Since a frequency range of pulse variation caused by trunk muscle contraction is on the order of 0.04 to 0.4 Hz, the above-described certain number of pulses may be on the order of 2 to 20 pulses. For example, a differential value can be obtained by obtaining the difference between a pulse interval value AAI(i) and a pulse interval value AAI(i+n) (n: on the order of 2 to 20) and dividing the difference by a time. Here, when the number of pulses corresponding to a differential interval is small (that is, when n is small), chances are high that low noise immunity is exhibited. When the number of pulses corresponding to a differential interval is large (that is, when n is large), chances are high that the processing speed decreases or a plurality of trunk muscle contraction movements are included in the differential interval. Preferably, the above-described certain number of pulses is 3 to 7 by taking such circumstances into consideration. In this manner, the differential processing unit 411 outputs differential-value data as change-component data of the pulse-interval data to the vibration-component removing unit 420.

The vibration-component removing unit 420 includes a positive-side peak detection unit 421, a positive-side peak holding unit 422, a negative-side peak detection unit 423, a subtraction unit 424, and an output unit 425. The vibration-component removing unit 420 generates vibration-component removal data by removing a component of pulse-interval data corresponding to periodic vibrations from change-component data on which the change-component acquisition unit 410 has performed differential processing. Since a pulse interval value caused by trunk muscle contraction decreases and then increases in a short period of time as described above, consecutive peaks, which are a negative peak and then a positive peak, appear in a differential waveform corresponding to this variation. Thus, a differential waveform of pulse-interval data caused by the above-described trunk muscle contraction is left by performing a process for removing consecutive peaks that occur in the order opposite to this order, that is, a waveform component in which a negative peak occurs after a positive peak in a consecutive manner.

The positive-side peak detection unit 421 determines the presence or absence of positive peaks in the above-described differential waveform, and causes, in the case where a positive peak is detected, the positive-side peak holding unit 422 to hold a positive-peak characteristic value regarding the positive peak.

The negative-side peak detection unit 423 detects whether or not a negative peak occurs before a certain number of pulses occurs after the positive peak has been detected by the positive-side peak detection unit 421. In the case where a negative peak is not detected before the certain number of pulses occurs after the positive peak has been detected, the positive-peak characteristic value held by the positive-side peak holding unit 422 is discarded.

In the case where a negative peak is detected by the negative-side peak detection unit 423 before the certain number of pulses occurs after the positive peak has been detected, the subtraction unit 424 performs subtraction processing for a negative-peak characteristic value regarding the negative peak in accordance with the positive-peak characteristic value held by the positive-side peak holding unit 422. For example, the positive-peak characteristic value is subtracted from the negative-peak characteristic value. Here, the positive-peak characteristic value and the negative-peak characteristic value may be respective peak values, or may also be differential values over time.

For example, as illustrated in FIG. 4, assume a case where a positive peak X having a positive-peak characteristic value x occurs at a time tx, and thereafter a negative peak Y having a negative-peak characteristic value y occurs at a time ty in a differential waveform. In the case where a time difference ty-tx is within a certain number-of-pulse range, the positive-peak characteristic value x is subtracted from the negative-peak characteristic value y. In the case where the time difference ty-tx is outside the certain number-of-pulse range, subtraction processing is not performed for the negative-peak characteristic value y. Note that the above-described certain number-of-pulse range is set on the basis of a number-of-pulse range corresponding to respiratory variation (on the order of 2 to 16 pulses), and is preferably on the order of 1 to 8 pulses.

A vibration-component removal process will be described in greater detail using FIG. 5. FIG. 5 illustrates a differential waveform similarly to as in FIG. 4, and the horizontal axis represents time. In FIG. 5, positive peaks X1 to X4 on the positive side and negative peaks Y1 to Y4 on the negative side are illustrated in order of time. Suppose that occurrence times are tx1 to tx4 and ty1 to ty4, and respective peak characteristic values are x1 to x4 and y1 to y4. In addition, suppose that time differences ty1−tx1, ty2−tx2, and ty3−tx3 are within the above-described certain number-of-pulse range, and a time difference ty4−tx4 is outside the certain number-of-pulse range.

Here, for the negative peak Y1 that has appeared immediately after the positive peak X1, a peak characteristic value y1−x1 is obtained. Since the peak characteristic value x1 is approximately equal to the peak characteristic value y1, a subtraction result for the peak characteristic value of the negative peak Y1 becomes substantially zero. Thus, the peak Y1 has been removed as a vibration component. Likewise, for the negative peak Y2 that has appeared immediately after the positive peak X2, a peak characteristic value y2−x2 is obtained. Since the peak characteristic value x2>the peak characteristic value y2, a subtraction result for the peak characteristic value of the negative peak Y2 takes a negative value (or may also be treated as zero). Thus, the peak Y2 has been removed as a vibration component. In addition, for the negative peak Y3 that has appeared immediately after the positive peak X3, a peak characteristic value y3−x3 is obtained. Since the peak characteristic value x3<the peak characteristic value y3, a subtraction result for the peak characteristic value of the negative peak Y3 does not become zero. Thus, the peak Y3 is not removed as a vibration component. For the negative peak Y4 that appears after the positive peak X4, since a time difference ty4−tx4 is outside the certain number-of-pulse range as described above, subtraction processing is not performed. That is, the negative peak Y4 is not also removed as a vibration component.

The output unit 425 outputs, to the variation-component extraction unit 430, differential data supplied from the differential processing unit 411 of the change-component acquisition unit 410 and subtraction data supplied from the subtraction unit 424. The output unit 425 may make, as necessary, a subtraction result obtained by the subtraction unit 424 match temporally to other data for which subtraction processing is not performed by the subtraction unit 424. In this manner, vibration-component removal data obtained by removing the component corresponding to periodic vibrations (differential values) from the pulse-interval data (differential values) is output from the vibration-component removing unit 420 to the variation-component extraction unit 430.

The variation-component extraction unit 430 includes an inversion unit 431 and a comparison unit 432, extracts a certain variation component from vibration-component removal data supplied from the vibration-component removing unit 420, determines the occurrence of trunk muscle contraction in accordance with the extracted variation component, and determines a time at which a certain variation component is given to be a trunk muscle contraction detection time.

The inversion unit 431 inverts vibration-component removal data supplied from the vibration-component removing unit 420, and outputs positive-side values of the inverted vibration-component removal data (hereinafter referred to as “inversion data”). As a result, the data Q illustrated in FIG. 2 is obtained.

The comparison unit 432 extracts peaks having values greater than or equal to a certain threshold from the inversion data. With reference to the data Q of FIG. 2, peaks p1, p2, p3, p4, and p6 having values greater than or equal to the certain threshold (AAI=0.1 in this example) are extracted, and a peak p5 having a value less than the threshold is not extracted among the peaks p1 to p6. Note that as illustrated in FIG. 3, peaks having values greater than or equal to the threshold do not occur in the data S, which is detection results obtained by performing the above-described processes on respiratory vibrations. This shows that, in the case where only respiratory variations exist in the pulse-interval data, variations caused by trunk muscle contraction are not detected as results of the above-described processes.

In this manner, the variation-component extraction unit 430 determines trunk muscle contraction in accordance with occurrence of a peak portion where a negative-side peak value of the vibration-component removal data is greater than or equal to the certain threshold, and outputs the time at which the peak portion has occurred as a detection time of the trunk muscle contraction. In the case where the time of the peak portion is delayed because of a filtering process or the like, the variation-component extraction unit 430 calculates the delay time, and outputs a time obtained by performing correction for the delay time as the detection time of the trunk muscle contraction.

Trunk muscle contraction detection results (the presence or absence of occurrence of trunk muscle contraction, the trunk muscle contraction detection time, and so forth) determined by the variation-component extraction unit 430 of the pulse-interval data analysis unit 40 are output to, for example, a display 50. The display 50 is, for example, a liquid crystal display (LCD) or the like. Note that the acquired pulse data, pulse-interval data, and trunk muscle contraction detection results are, for example, accumulated and stored in the above-described RAM or the like, and may be output to a personal computer (PC) or the like and confirmed after completion of measurement. In addition, the trunk muscle contraction detection results may also be output as voice or sound from a speaker 55. A beeping sound, a chime, a spoken instruction, or the like may be output from the speaker 55 at the time of detection of trunk muscle contraction. Furthermore, the above-described detection results may also be transmitted to, for example, a PC, a smartphone, or the like via a communication unit 60, and may be displayed.

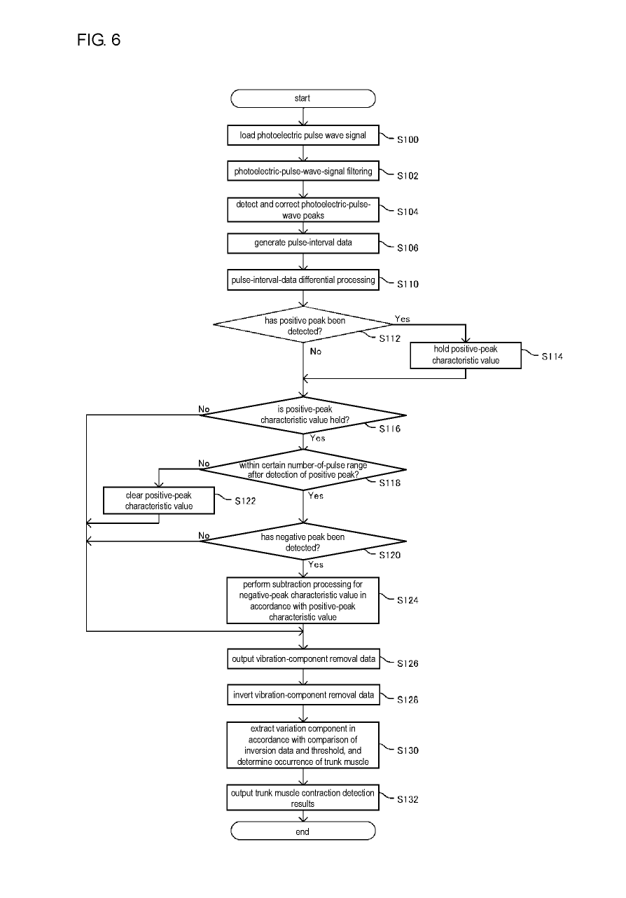

Next, an operation of the trunk muscle contraction detection apparatus 1 will be described with reference to FIG. 6. First, in step S100, a photoelectric pulse wave signal (photoelectric pulse wave waveform) detected by the photoelectric pulse wave sensor 20 is loaded.

Next, in step S102, a filtering process is performed on the photoelectric pulse wave signal loaded in step S100. In addition, the photoelectric pulse wave signal is subjected to second-order differentiation, whereby acquiring an acceleration pulse wave. Next, in step S104, peaks of the photoelectric pulse wave signal (acceleration pulse wave signal) are detected. Then, for each of the detected peaks, information on a peak time, a peak amplitude, and the like is stored. Furthermore, for each peak of the photoelectric pulse wave signal (acceleration pulse wave), a delay time (the amount of shift) is obtained, and the peak of the photoelectric pulse wave signal (acceleration pulse wave) is corrected in accordance with the obtained delay time. Note that since the correction method for each peak has been described above, detailed description thereof is omitted here. In step S106, pulse-interval data is generated by collecting the corrected peaks of the photoelectric pulse wave. Here, the pulse-interval data generation unit 330 of the pulse-interval acquisition unit 30 generates pulse-interval data, and outputs the pulse-interval data to the pulse-interval data analysis unit 40.

In step 110, the differential processing unit 411 of the change-component acquisition unit 410 differentiates the pulse-interval data, in which pulse interval values are arranged in time series, using the number of pulses on the order of 2 to 20 as a differential interval. For example, the difference between a pulse interval value AAI(i) and a pulse interval value AAI(i+n) (n: on the order of 2 to 20, preferably 3 to 7) is obtained, and the difference may be used as a differential value. Then, the change-component acquisition unit 410 outputs differential waveform data serving as change-component data to the vibration-component removing unit 420.

Next, in steps S112 to S126, the vibration-component removing unit 420 generates vibration-component removal data by removing a component of the pulse-interval data corresponding to periodic vibrations from the change-component data on which the change-component acquisition unit 410 has performed differential processing. As described above, the vibration-component removing unit 420 performs a process for removing a waveform component in which a negative peak occurs after a positive peak in a consecutive manner from the change-component data. These steps are performed by the positive-side peak detection unit 421, the positive-side peak holding unit 422, the negative-side peak detection unit 423, the subtraction unit 424, the output unit 425, and the CPU.

In step S112, the positive-side peak detection unit 421 determines the presence or absence of a positive peak in the change-component data. In the case where a positive peak is detected (step S112, Yes), the positive-side peak holding unit 422 holds a positive-peak characteristic value in step S114. After step S114 or in the case where a positive peak has not been detected (step S112, No), the process proceeds to step S116.

In step S116, it is determined whether or not a positive-peak characteristic value is held by the positive-side peak holding unit 422. In the case where a positive-peak characteristic value is held by the positive-side peak holding unit 422 (step S116, Yes), the process proceeds to step S118. Otherwise, the process proceeds to step S126.

In step S118, the negative-side peak detection unit 423 determines whether or not the number of pulses that have occurred after detection of the positive peak is within a certain number-of-pulse range. In the case where the number of pulses that have occurred is within the certain number-of-pulse range (step S118, Yes), the process proceeds to step S120. In contrast, in the case where the number of pulses that have occurred is outside the certain number-of-pulse range (step S118, No), the positive-peak characteristic value held by the positive-side peak holding unit 422 is discarded in step S122.

In step S120, the negative-side peak detection unit 423 determines the presence or absence of a negative peak in the change-component data. In the case where a negative peak has been detected (step S120, Yes), the process proceeds to step S124. In the case where a negative peak has not been detected (step S120, No), the process proceeds to step S126.

In step S124, the subtraction unit 424 performs subtraction processing for a negative-peak characteristic value in accordance with the positive-peak characteristic value held by the positive-side peak holding unit 422. For example, the positive-peak characteristic value is subtracted from the negative-peak characteristic value. As a result, the component of a negative peak that appears within the certain number-of-pulse range from the positive peak is removed from the change-component data (that is, differential waveform data).

In step S126, the output unit 425 outputs data on which differential processing has been performed in step S110 and data on which subtraction processing has been performed in step S124. As a result, vibration-component removal data is output to the variation-component extraction unit 430 in a state where the subtraction result obtained in step S124 is made to, as necessary, match temporally to other data that is not subjected to step S124.

In steps S128 to S130, the variation-component extraction unit 430 extracts a certain variation component from the vibration-component removal data supplied from the vibration-component removing unit 420, and determines the occurrence of trunk muscle contraction in accordance with the extracted variation component.

In step S128, the inversion unit 431 inverts the vibration-component removal data supplied from the vibration-component removing unit 420, and outputs inversion data which is a positive-side value of the inverted vibration-component removal data.

In step S130, the comparison unit 432 compares the inversion data with a certain threshold, determines the occurrence of trunk muscle contraction by extracting inversion data having a value greater than or equal to the threshold as a peak, and determines a time at which a certain variation component is given to be a trunk muscle contraction detection time.

In step S132, the display 50 and the like output, by for example performing display, trunk muscle contraction detection results (the presence or absence of occurrence of trunk muscle contraction, the trunk muscle contraction detection time, and so forth) determined in step S130.

Note that a configuration for substantially removing a negative peak that appears immediately after a positive peak is used as a configuration for removing a periodical vibration component in the present embodiment; however, an adaptive filter may also be used that adaptively removes a vibration component corresponding to the number of pulses for respiratory variation. In addition, in the present embodiment, a configuration has been illustrated with which the vibration-component removal process is performed by the vibration-component removing unit 420 after differential processing has been performed by the change-component acquisition unit 410; however, a configuration may also be used with which differential processing is performed after the vibration-component removal process. In addition, in the present embodiment, it has been described above that the pulse sensor (photoelectric pulse wave sensor) 20, the pulse-interval data acquisition unit 30, the pulse-interval data analysis unit 40, and the like are integrally formed; however, the pulse-interval data analysis unit 40 may be prepared separately from the pulse-interval data acquisition unit 30. In this case, the pulse-interval data supplied from the pulse-interval data acquisition unit 30 is transmitted to the pulse-interval data analysis unit 40 using wired communication or wireless communication.

As described above, since the periodical vibration component caused by breathing and the like is removed from the pulse-interval data with the configuration according to the present embodiment, pulse variations caused by trunk muscle contraction may be assuredly extracted. In addition, since the time from when trunk muscle contraction has occurred to when detection results are output substantially requires only approximately a few pulses for performing differential processing, detection results can be obtained timely in a semi-real time manner.

### Second Embodiment

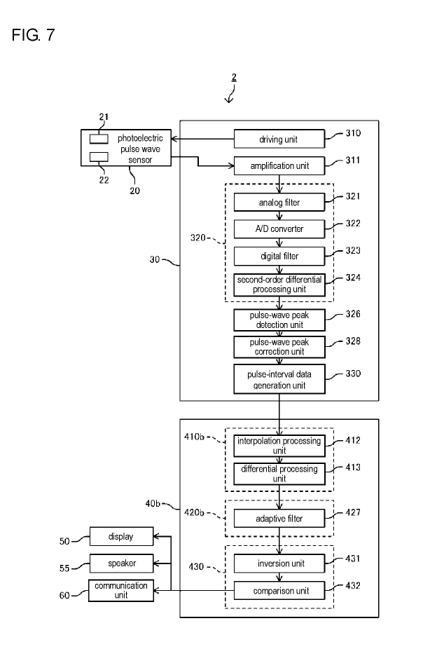

The configuration of a trunk muscle contraction detection apparatus 2 according to a second embodiment will be described using FIG. 7. FIG. 7 is a block diagram illustrating the configuration of the trunk muscle contraction detection apparatus 2. Note that structural elements substantially the same as those of the trunk muscle contraction detection apparatus 1 of the first embodiment will be denoted by the same reference numerals, and description thereof will be omitted. The trunk muscle contraction detection apparatus 2 includes a pulse-interval data analysis unit 40b. The pulse-interval data analysis unit 40b includes a change-component acquisition unit 410b, a vibration-component removing unit 420b, and the variation-component extraction unit 430. That is, the change-component acquisition unit 410b and the vibration-component removing unit 420b differ from the change-component acquisition unit 410 and the vibration-component removing unit 420 of the first embodiment. The change-component acquisition unit 410b includes an interpolation processing unit 412 and a differential processing unit 413.

The interpolation processing unit 412 performs an interpolation process on pulse-interval data in which pulse intervals are arranged in time series. Here, the interpolation process is, for example, spline interpolation. As a result, pulse-interval data may be cut out at constant time intervals (for example, intervals of 0.01 seconds). This interpolation process makes it possible to process data as a function of frequency in subsequent processes.

The differential processing unit 413 differentiates interpolated pulse-interval data at certain time intervals. Here, since a frequency range of pulse variation generated by trunk muscle contraction is on the order of 0.04 to 0.4 Hz, a time interval for differentiation is preferably on the order of 1.25 to 12.5 seconds.

The vibration-component removing unit 420b includes an adaptive filter 427. The adaptive filter 427 is configured to remove periodical vibrations of waveform data supplied from the change-component acquisition unit 410b. Since the periodical vibrations to be removal targets have a frequency range of the order of 0.1 to 0.5 Hz corresponding to respiratory variation, the adaptive filter 427 makes its transfer function self-adapted in accordance with an optimization algorithm such that the frequency component of this range is removed. As a result, the pulse component that does not periodically vary due to the above-described frequencies is extracted as vibration-component removal data. Note that the adaptive filter 427 is used as the vibration-component removing unit 420b in the present embodiment; however, in the case where the frequencies of a periodical vibration component to be removed have been known in advance (for example, in the case where a pulse-interval data analysis unit 40b specifically designed for a specific subject is generated), for example, a frequency filter having a relatively narrow cutoff frequency range may be used.

In this manner, the vibration-component removal data supplied from the vibration-component removing unit 420b is input to the variation-component extraction unit 430, and a data extraction process substantially the same as that of the first embodiment is performed in the variation-component extraction unit 430. Trunk muscle contraction detection results (the presence or absence of occurrence of trunk muscle contraction, a trunk muscle contraction detection time, and so forth) determined by the variation-component extraction unit 430 are output to the display 50 or the speaker 55. In addition, the above-described detection results may also be transmitted to, for example, a PC, a smartphone, or the like via the communication unit 60, and may be displayed.

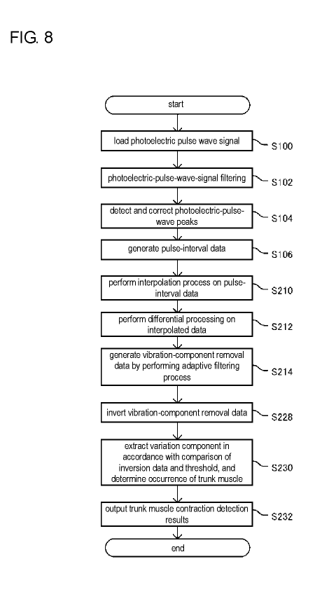

Next, an operation of the trunk muscle contraction detection apparatus 2 according to the present embodiment will be described with reference to FIG. 8. The process in steps S102 to S106 is substantially the same as the process in steps S102 to S106 of the first embodiment illustrated in FIG. 6, and thus description thereof will be omitted.

In step S210 after step S106, the interpolation processing unit 412 applies an interpolation process such as spline interpolation to pulse-interval data in which pulse intervals are arranged in time series, and cuts out the pulse-interval data at constant time intervals, for example, at intervals of the order of 0.01 seconds.

In step S212, the differential processing unit 413 differentiates the interpolated pulse-interval data at time intervals of the order of 1.25 to 12.5 seconds, and outputs this as change-component data to the vibration-component removing unit 420b.

In step S214, the adaptive filter 427 removes, for example, periodical vibrations on the order of 0.1 to 0.5 Hz from the change-component data input from the change-component acquisition unit 410b, and extracts a pulse component that does not periodically vary due to the above-described frequencies as vibration-component removal data.

Steps S228 to S232 after step S214 are substantially the same as steps S128 to S132 of the first embodiment. That is, the inversion unit 431 inverts the vibration-component removal data and outputs inversion data in step S228. In step S230, the comparison unit 432 compares the inversion data with a certain threshold, determines the occurrence of trunk muscle contraction by extracting inversion data having a value greater than or equal to the threshold as a peak, and determines a time at which a certain variation component is given to be a trunk muscle contraction detection time. Then, in step S232, the display 50 and the like output, by for example performing display, trunk muscle contraction detection results (the presence or absence of occurrence of trunk muscle contraction, the trunk muscle contraction detection time, and so forth) determined in step S230.

As described above, with the configuration of the present embodiment, an interpolation process makes it possible to process pulse-interval data on the frequency axis, and the degree of freedom in the design of a process for vibration component removal is improved. In addition, it becomes easier to capture vibration characteristics by performing an interpolation process on pulse-interval data having a short vibration period corresponding to, for example, about two to three pulses, and thus the accuracy of vibration component removal may be improved.

### Third Embodiment

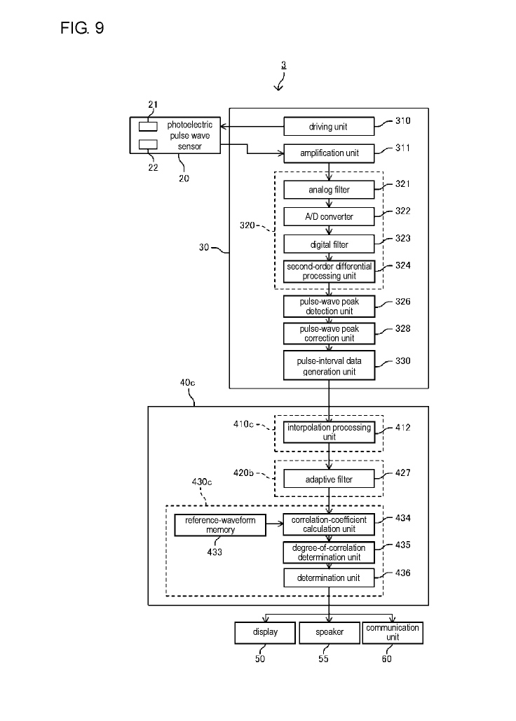

The configuration of a trunk muscle contraction detection apparatus 3 according to a third embodiment will be described using FIG. 9. FIG. 9 is a block diagram illustrating the configuration of the trunk muscle contraction detection apparatus 3. Note that structural elements substantially the same as those of the trunk muscle contraction detection apparatus 2 of the second embodiment will be denoted by the same reference numerals, and description thereof will be omitted. The trunk muscle contraction detection apparatus 3 includes a pulse-interval data analysis unit 40c. The pulse-interval data analysis unit 40c includes a change-component acquisition unit 410c, the vibration-component removing unit 420b, and a variation-component extraction unit 430c. That is, the configurations of the change-component acquisition unit 410c and variation-component extraction unit 430c differ from those of the change-component acquisition unit 410b and the variation-component extraction unit 430 of the second embodiment.

The change-component acquisition unit 410c includes the interpolation processing unit 412. The interpolation processing unit 412 performs an interpolation process on pulse-interval data in which pulse intervals are arranged in time series. Here, the interpolation process is, for example, spline interpolation. As a result, pulse-interval data may be cut out at constant time intervals (for example, intervals of 0.01 seconds). This data on which the interpolation process has been performed is processed as a function of frequency.

Similarly to as in the second embodiment, the vibration-component removing unit 420b includes the adaptive filter 427. That is, the adaptive filter 427 generates vibration-component removal data by removing periodical vibrations (0.1 to 0.5 Hz) of waveform data supplied from the change-component acquisition unit 410c. The vibration-component removal data supplied from the vibration-component removing unit 420b is input to the variation-component extraction unit 430c.

The variation-component extraction unit 430c includes a reference-waveform memory 433, a correlation-coefficient calculation unit 434, a degree-of-correlation determination unit 435, and a determination unit 436.

The reference-waveform memory 433 stores a reference waveform serving as a model for pulse variations to be detection targets. A model into which pulse variations that occur at the times of trunk muscle movement are converted is used as this reference waveform. As a simple model, a model obtained by approximating the variation in the number of pulses to a square wave, a triangular wave, or the like may be used.

The correlation-coefficient calculation unit 434 calculates a correlation coefficient between vibration-component removal data and a reference waveform stored in the reference-waveform memory 433. The degree-of-correlation determination unit 435 outputs a detection signal and the degree of correlation in the case where the correlation coefficient calculated by the correlation-coefficient calculation unit 434 exceeds a certain value.

The determination unit 436 extracts, in the case where a detection signal has been input, a variation component in accordance with the degree of correlation, determines the occurrence of trunk muscle contraction, and determines the time at which the variation component is given to be a trunk muscle contraction detection time. In this manner, trunk muscle contraction detection results (the presence or absence of occurrence of trunk muscle contraction, the trunk muscle contraction detection time, and so forth) determined by the variation-component extraction unit 430 are output to the display 50 or the speaker 55. In addition, the above-described detection results may also be transmitted to, for example, a PC, a smartphone, or the like via the communication unit 60, and may be displayed.

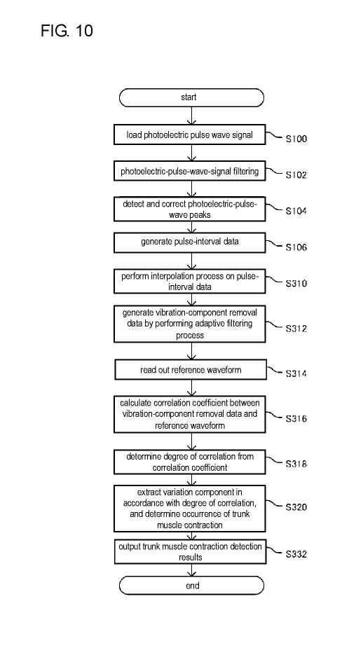

Next, an operation of the trunk muscle contraction detection apparatus 3 according to the present embodiment will be described with reference to FIG. 10. The process in steps S102 to S106 is substantially the same as the process in steps S102 to S106 of the first embodiment illustrated in FIG. 6, and thus description thereof will be omitted.

In step S310 after step S106, the interpolation processing unit 412 applies an interpolation process such as spline interpolation to pulse-interval data in which pulse intervals are arranged in time series, and cuts out the pulse-interval data at constant time intervals, for example, at intervals of the order of 0.01 seconds.

In step S312, the adaptive filter 427 removes, for example, periodical vibrations on the order of 0.1 to 0.5 Hz from the change-component data input from the change-component acquisition unit 410c, and extracts a pulse component that does not periodically vary due to the above-described frequencies as vibration-component removal data.

In step S314, a reference waveform serving as a model for pulse variations to be detection targets is read out from the reference-waveform memory 433.

In step S316, the correlation-coefficient calculation unit 434 calculates a correlation coefficient between the vibration-component removal data and the reference waveform. In step S318, the degree-of-correlation determination unit 435 outputs, in the case where the correlation coefficient calculated in step S316 exceeds a certain value, a detection signal and the degree of correlation.

In step S320, the determination unit 436 extracts, in the case where the detection signal has been input, a variation component in accordance with the degree of correlation, determines the occurrence of trunk muscle contraction, and determines the time at which the variation component is given to be a trunk muscle contraction detection time. Then, in step S332, the display 50 and the like output, by for example performing display, trunk muscle contraction detection results (the presence or absence of occurrence of trunk muscle contraction, the trunk muscle contraction detection time, and so forth) determined in step S320.

Note that the configuration using an adaptive filter in the vibration-component removal process has been described in the present embodiment; however, a configuration may also be used with which the wavelet transform is performed, a periodical vibration component is removed from the converted data, and a variation component caused by trunk muscle contraction is extracted from the data obtained after the period-vibration-component removal. In addition, as a variation-component extraction process, a configuration has been described with which a correlation coefficient between vibration-component removal data and a reference waveform is calculated. However, a configuration may also be used with which differential processing is performed at certain time intervals, a peak that is a negative peak having a value greater than or equal to a certain threshold is extracted, and the time of this peak is output as a trunk muscle contraction detection time.

As described above, even with the configuration of the present embodiment, similarly to as in the second embodiment, it is possible to process pulse-interval data in a frequency domain, and the degree of freedom in the design of a process for vibration component removal is improved. In addition, it becomes easier to capture vibration characteristics by performing an interpolation process on pulse-interval data having a short vibration period corresponding to, for example, about two to three pulses, and thus the accuracy of vibration component removal may be improved.

### REFERENCE SIGNS LIST

1, 2, 3 the trunk muscle contraction detection apparatus
20 pulse sensor
30 pulse-interval data generation unit
40, 40b, 40c pulse-interval data analysis unit
410, 410b, 410c change-component acquisition unit
411, 413 differential processing unit
412 interpolation processing unit
420, 420b vibration-component removing unit
421 positive-side peak detection unit
422 positive-side peak holding unit
423 negative-side peak detection unit
424 subtraction unit
425 output unit
430, 430c variation-component extraction unit
431 inversion unit
432 comparison unit
433 reference-waveform memory
434 correlation-coefficient calculation unit
435 degree-of-correlation determination unit
436 determination unit

1. A trunk muscle contraction detection apparatus comprising:
a processor coupled to the sensor and configured to:
generate pulse-interval data from a biological signal,
acquire change-component data that represents a change component of the pulse-interval data, the change component relating to pulse intervals,
remove a vibration component from the change-component data, the vibration component corresponding to periodic vibrations in the pulse-interval data,
extract a variation component from the vibration-component removal data, and
determine trunk muscle contractions based on the extracted variation component.

2. The trunk muscle contraction detection apparatus according to claim 1, wherein the variation component is a component corresponding to a change waveform where an upward peak is detected after a downward peak in a waveform representing the pulse-interval data.

3. The trunk muscle contraction detection apparatus according to claim 1, wherein the vibration component is based on respiratory variation.

4. The trunk muscle contraction detection apparatus according to claim 1, wherein the processor is further configured to interpolate the pulse-interval data and arrange data relating to the pulse-interval data at constant time intervals.

5. The trunk muscle contraction detection apparatus according to claim 4, wherein the processor is further configured to remove the vibration component from the interpolated pulse-interval data.

6. The trunk muscle contraction detection apparatus according to claim 1, further comprising a sensor configured to output the biological signal.

7. The trunk muscle contraction detection apparatus according to claim 1, further comprising a signal processor including:
an amplification circuit configured to amplify the biological signal;
at least one filter configured to remove noise components from the amplified signal; and
a second-order differential processing unit configured to generate an acceleration pulse wave based on a signal output from the at least one filter.

8. The trunk muscle contraction detection apparatus according to claim 7, wherein the processor is further configured to:
detect a rising edge of the acceleration pulse wave,
calculate a time delay of the acceleration pulse wave,
correct, based on the calculated time delay, a peak of the acceleration pulse wave, and
generate the pulse-interval data by collecting corrected peaks of the biological signal.

9. The trunk muscle contraction detection apparatus according to claim 1, wherein the change-component data comprises a differential waveform.

10. The trunk muscle contraction detection apparatus according to claim 9, wherein the processor is further configured to remove the vibration component from the differential waveform by:
detecting a positive peak in the differential waveform;
detecting whether a negative peak occurs in the differential waveform after a predetermined pulses after detecting the positive peak;
if the negative peak is detected in the differential waveform after the predetermined pulses, subtracting a value corresponding to the positive peak from a value corresponding to the negative peak to generate the vibration-component removal data.

11. A method for detecting trunk muscle contractions, the method comprising:
generating, by a processor, pulse-interval data from a biological signal;
acquiring, by the processor, change-component data that represents a change component of the pulse-interval data, the change component relating to pulse intervals;
removing, by the processor, a vibration component from the change-component data, the vibration component corresponding to periodic vibrations in the pulse-interval data;
extracting, by the processor, a variation component from the vibration-component removal data; and
determining, by the processor, trunk muscle contractions based on the extracted variation component.

12. The method according to claim 11, wherein the variation component is a component corresponding to a change waveform where an upward peak is detected after a downward peak in a waveform representing the pulse-interval data.

13. The method according to claim 11, wherein the vibration component is based on respiratory variation.

14. The method to claim 11, further comprising:
interpolating, by the processor, the pulse-interval data; and
arranging, by the processor, data relating to the pulse-interval data at constant time intervals.

15. The method according to claim 14, further comprising removing, by the processor, the vibration component from the interpolated pulse-interval data.

16. The method according to claim 11, further comprising generating, by a sensor, the biological signal.

17. The method according to claim 11, further comprising:
amplifying, by an amplification circuit, the biological signal;
removing, by at least one filter, noise components from the amplified signal; and
generating, by a second-order differential processing unit, an acceleration pulse wave based on a signal output from the at least one filter.

18. The method according to claim 17, further comprising:
detecting, by the processor, a rising edge of the acceleration pulse wave;
calculating, by the processor, a time delay of the acceleration pulse wave;
correcting, based on the calculated time delay, a peak of the acceleration pulse wave; and
generating, by the processor, the pulse-interval data by collecting corrected peaks of the biological signal.

19. The method according to claim 11, wherein the change-component data comprises a differential waveform.

20. The method according to claim 19, further comprising removing the vibration component from the differential waveform by:
detecting a positive peak in the differential waveform;
detecting whether a negative peak occurs in the differential waveform after a predetermined pulses after detecting the positive peak;
if the negative peak is detected in the differential waveform after the predetermined pulses, subtracting a value corresponding to the positive peak from a value corresponding to the negative peak to generate the vibration-component removal data.
